(19) United States
(12) Plant Patent Application Publication
(10) Pub. No.: US 2002/0184680 P1
(43) Pub. Date: Dec. 5, 2002

(54) Chrysanthemum plant named 'GEDI ONE OST'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,601

(22) Filed: Jun. 4, 2001

(51) Int. Cl. A01H 5/00

(52) U.S. Cl. PLT/289

(57) ABSTRACT

A new and distinct Chrysanthemum plant variety characterized by a daisy type inflorescence, consistent flowering response to short days, blooming consistently after 49 days of short day length, free branching habit, compact growth habit, and highly floriferous plants.

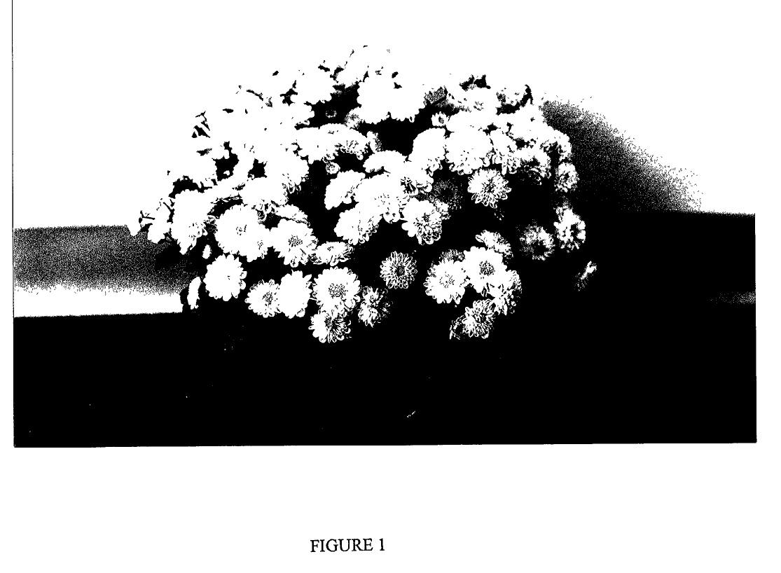

### BACKGROUND OF THE INVENTION

The present invention relates to a new and distinct cultivar of Chrysanthemum plant, botanically known as Dendranthema morifolium and herein after referred to by the cultivar name ‘GEDI ONE OST.’

This new cultivar originated from hybridization of a female parent Chrysanthemum cultivar ‘Ballino’ (U.S. Plant patent application Ser. No. 09/550,159) with a male parent Chrysanthemum cultivar ‘Sunny Tripoli’ (unpatented in the United States). The new cultivar was discovered by the inventor in October 1996.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE INVENTION

The cultivar ‘GEDI ONE OST’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘GEDI ONE OST.’ These characteristics in combination distinguish ‘GEDI ONE OST’ as a new and distinct Chrysanthemum:

1. Daisy type inflorescence;

2. Consistent flowering response to short days, blooming consistently after 49 days of short day length;

3. Free branching habit;

4. Compact growth habit; and

5. Highly floriferous plants.

Plants of the new cultivar are similar to plants of the parent cultivar, ‘Ballino,’ in most horticultural characteristics, however plants of the new cultivar differed from plants of the cultivar ‘Ballino’ in the following characteristics:

1. The new cultivar naturally blooms 1 week later; and

2. The new cultivar has larger diameter blooms.

Plants of the new cultivar are similar to plants of the parent cultivar, ‘Sunny Tripoli,’ in most horticultural characteristics, however plants of the new cultivar differed from plants of the cultivar ‘Sunny Tripoli’ in the following characteristics:

1. The new cultivar has more ray florets;

2. The new cultivar has a more round, uniform plant shape; and

3. The new cultivar naturally blooms 1 week later.

The new cultivar can be compared to the Chrysanthemum cultivar ‘Yellow Tripoli’ (disclosed in Plant Pat. No. 11,725). However plants of the new Chrysanthemum differed from the plants of the cultivar ‘Yellow Tripoli’ in the following characteristics:

1. The new cultivar has more ray florets;

2. The new cultivar has a more round plant shape and greater branching;

3. The new cultivar has a shorter internode length; and

4. The new cultivar has more blooms per flowering branch.

### BRIEF DESCRIPTION OF THE PHOTOGRAPHS

The photograph shows a plant of ‘GEDI ONE OST’ grown in an 8-inch container. A single cutting was used in the container.

### DETAILED BOTANICAL DESCRIPTION

In the following description, color references are made to The Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70° to 85° during the day. Measurements and numerical values represent averages of typical flowering types.

Botanical classification: Dendranthema morifolium cultivar ‘GEDI ONE OST’

Commercial classification: Garden-type Chrysanthemum

Parentage: Hybridization of female parent Chrysanthemum cultivar ‘Ballino’ (U.S. Plant patent application Ser. No. 09/550,159) with male parent Chrysanthemum cultivar ‘Sunny Tripoli’ (unpatented in the United States)

### Propagation

Time to Rooting: 7 to 14 days at approximately 21° C.

Rooting habit: Fine, fibrous

### Plant

Growth Habit: Mounding herbaceous perennial

Height: Approximately 24.5 cm

Growth rate: Moderate

Branching Characteristics: Free Branching

Length of Lateral Branches: Approximately 21 cm

Number of leaves per lateral branch: Approximately 19

### Foliage

Leaf:

Arrangement.—Altemnate Single

Average length.—Approximately 4.9 cm

Average Width.—Approximately 0.3 cm

Shape of blade.—Ovate

Apex.—Acuminate

Base.—Attenuate

Attachment.—Stalked

Margin.—Serrate

Texture of top surface.—Lightly pubescent

Texture of bottom surface.—Lightly pubescent

Color.—young foliage upper side: Near 147A young foliage under side: Near 147A mature foliage upper side: Near 147A mature foliage under side: Near 147A venation upper side: Near 148B venation under side: Near 147B

Petiole:

Average length.—Approximately 1.3 cm

### Bloom

Inflorescence:

Flowering habit.—Induced by darkness period greater than 13.5 hours, approximately 49 days of appropriate day length required to induce and develop blooms

Inflorescence form.—Daisy

Natural flowering season.—Approximately the last 2 weeks in September Number of inflorescences per stem: Approximately 15-30

Inflorescence diameter.—Approximately 3.5 cm

Inflorescence depth.—Approximately 0.8 cm

Disc diameter.—Approximately 1 cm

Inflorescence longevity on plant.—Approximately 26-29 days

Persistence.—Persistent

Ray Florets:

Appearance.—Matte

Texture.—Smooth

Average number per flower.—Approximately 90 to 100

Shape.—Cylindric

Margin.—Entire

Apex.—Obtuse

Length.—Approximately 1 cm

Width.—Approximately 0.6 cm

Color.—upper surface atfirst opening: Near 14A upper surface at maturity: Near 9A upper surface atfading: Near 9B under surface atfirst opening: Near 14A under surface at maturity: Near 9A under surface at fading: Near 9B

Disc florets:

Appearance.—Shiny

Texture.—Smooth

Average number per flower.—Approximately 80 to 90

Shape.—Cylindric

Apex.—Obtuse

Average length.—Approximately 0.5 cm

Average width.—Approximately 0.1 cm

Peduncle:

Length.—at terminal end (shortest): Approximately 2.5 cm at lateral end (longest): Approximately 4.8 cm

Strength.—Moderate

Color.—Near 147B

Habit.—Erect

Inflorescence bud:

Length.—Approximately 0.6 cm

Diameter.—Approximately 0.7 cm

Form.—Globular

Color.—Near 5A

Sepals:

Appearance.—Matte

Texture.—Lightly pubescent

Number.—Approximately 30 to 40

Shape.—Ovate

Margin.—Entire

Apex.—Acute

Color.—upper side: Near 137B underside: Near 137B

### Reproductive Organs

Ray Florets:

Pistillate.—Number ofpistils per flower: 1 Stigma shape: 2 branched Stigma color: Near 21A Stigma length: Approximately 0.1 cm

Disc Florets:

Pistillate.—Number of pistils per flower: 1 Stigma shape: Cylindric

Staminate.—Number of stamens per flower: Approximately 5 Anther shape: Tubular Anther color: Near 21A Pollen color: Near 21A

What is claimed is:

1. A new and distinct variety of Chrysanthemum plant named ‘GEDI ONE OST’ as herein illustrated and described.
